(19) United States
(12) Patent Application Publication
(10) Pub. No.: US 2007/0255149 A1
(43) Pub. Date: Nov. 1, 2007

(54) Apparatus for regulating blood flow to treat cardiac abnormalities and methods of use

(76) Inventor: William M. DeCampli, Winter Park, FL (US)

Correspondence Address: LUCE, FORWARD, HAMILTON & SCRIPPS LLP, 11988 EL CAMINO REAL, SUITE 200, SAN DIEGO, CA, 92130, US

(21) Appl. No.: 11/405,085

(22) Filed: Apr. 17, 2006

(51) Int. Cl. A61B 5/04

(52) U.S. Cl. 600/509

(57) ABSTRACT

Apparatus and methods for controlling blood flow through an aortopulmonary graft subsequent to completion of a first stage operation to palliate hypoplastic left heart syndrome and other cardiac abnormalities are provided, wherein an implantable cuff is applied to the graft, the cuff including an expandable element that is selectively actuated to cause constriction of the graft during diastole, responsive to detected cardiac activity, to reduce diastolic pulmonary runoff.

### FIELD OF THE INVENTION

The present invention relates generally to apparatus for regulating blood flow patterns within a patient to treat cardiac abnormalities. More specifically, the present invention relates to apparatus for regulating blood flow through an aortopulmonary shunt using counterpulsation techniques to reduce diastolic pulmonary runoff.

### BACKGROUND OF THE INVENTION

Hypoplastic left heart syndrome (HLHS) occurs in one in five thousand live births. It is typically characterized by an underdeveloped or non-existent left ventricle, narrowed aorta and underdeveloped or absent aortic valve. It is one of the most common heart anomalies resulting in a single chambered heart. Left untreated, babies suffering from this cardiac defect die within the first year of life. In the 1980's a series of staged operations were designed to treat this disorder, which progressively palliate the defect. Typically, the three stages of operations are referred to as the Norwood operation, bidirectional Glenn operation and Fontan operation. Initially, the Norwood operation reduced one-year mortality to about 50%—a significant advance for such patients. With further improvements in diagnosis, surgical treatments and intensive care management, the mortality rate has decreased to about 20% in the first year of life.

During a typical Norwood operation, the right and left pulmonary arteries are disconnected from the pulmonary artery trunk. A graft then is used to enlarge the underdeveloped aortic arch and join the reformed aortic arch to the pulmonary artery trunk. A shunt, typically referred to as a Blalock-Taussig shunt, then is connected from the innominate artery (originating from the aortic arch) to the pulmonary arteries. At the same time, the atrial septum is removed, so that oxygenated blood returning through the pulmonary veins mixes with deoxygenated blood in the right atrium. In this manner, blood exiting the single ventricle is directed into the patient's body, with a portion of the blood being diverted via the shunt to the lungs for oxygenation.

Following completion of the Norwood operation, an abnormal circulatory pattern persists, peculiar to the hemodynamics of single ventricle blood flow. In particular, because blood pumped into the aorta is only partially oxygenated, care must be taken when sizing and placing the shunt so that blood is partitioned with approximately one half directed to the lungs and the remainder directed to the body to deliver oxygen and nutrients. Inadequate physiological control over this partitioning results in either too little or too much blood being directed to the lungs; either extreme is fatal.

While the flow characteristics of the Blalock Taussig shunt determine the amount of blood directed to the lungs, the shunt alone cannot regulate partition of blood flow between the lungs and the rest of the body. Substantial mortality and morbidity therefore plague infants with this circulation, particularly within the first twenty-four to seventy-two hours after the first stage operation.

A substantial contributor to the morbidity and mortality directly relates to the hemodynamics of the shunt flow. The magnitude of flow through the shunt depends on a number of shunt characteristics, such as diameter, length, shunt orientation, vessel of origin, and the quality of the proximal and distal anastomoses (i.e., suture connections). If the flow is too great, the infant may succumb to congestive heart failure. If the flow is insufficient, the infant will become hypoxemic (i.e., suffer from a lack of oxygen). Additionally, during the diastolic phase of the cardiac cycle, blood may be siphoned through the shunt from the aortic arch to the pulmonary arteries. This in turn lowers the pressure in the aorta and reduces flow through the coronary arteries, potentially causing ventricular ischemia, cardiac dysfunction or even sudden catastrophic cardiac failure.

In approximately the year 2000, a modification of the Norwood operation was suggested with the aim of improving the post-operative hemodynamics and survival. In this modification, known as the “Sano variant,” a shunt is connected directly between the right ventricle and the pulmonary vessels, rather than originating from the aortic arch. Accordingly, rather than having blood flow through the shunt to the lungs both in systole and in diastole, as in the standard shunt, in the Sano variant, blood flows through the shunt only in systole.

In diastole, some blood actually flows backwards from the pulmonary vessel to the right ventricle. Experience reported using the Sano variant at selected institutions show that method has improved early post-operative (30 day) survival of patients with HLHS.

In the Sano variant, pulsatile flow is driven directly from the right ventricle, thus enhancing forward flow. Second, the shunts used are usually 1.5-3 mm larger in diameter than those used in a typical Norwood operation, ensuring more systolic blood flow in the absence of diastolic blood flow. Third, and perhaps most importantly, the location of the Sano shunt and the absence of diastolic blood flow prevents the shunt from siphoning blood from the aortic arch during diastole.

Most coronary blood flow occurs in diastole, or during ventricular filling. As noted previously, in a conventional Norwood operation, the shunt may siphon blood from the ascending aorta during diastole, since pulmonary resistance is much lower than systemic resistance. Heart muscle injury from lack of coronary blood flow has been documented in infants with this circulatory pattern, and is likely a substantial contributor to the instability experienced by infants in the early post-operative period.

Such “diastolic runoff” in the pulmonary vessels is most visibly manifested by a decrease in diastolic blood pressure. Whereas the normal diastolic pressure in a newborn is 35-40 mm Hg, the pressure is typically 10 mm lower in the presence of a conventional aortopulmonary shunt. A pressure below 20 mm Hg compromises coronary blood flow in the neonate.

Although the Sano variant provides substantial benefits, it is not without problems. Most importantly, many surgeons have found it difficult to position and suture the shunt in place so as to avoid kinking or distortion of the shunt or of the branch pulmonary vessels. Between the first and second stage operations to palliate HLHS, a high rate of unintended reintervention has been found necessary to treat pulmonary vessel distortion, and narrowing of the shunt near its connection to the right ventricle. Additionally, an incision directly into the single pumping heart chamber is necessary to position the shunt. The long-term effects of this incision continue to be of concern to surgeons. Lastly, studies of the longer-term (>30 day) hemodynamics of the Sano variant and outcome of the patients have failed to demonstrate a long-term advantage. For these reasons, the Sano variant has not become widely accepted.

Given the aforementioned problems, it would be desirable to be able to preserve the original (“standard”) configuration of the Norwood reconstruction, while at the same time providing a means to adjust flow within the shunt to reduce or eliminate “diastolic runoff”, thus achieving the same physiological advantage of the Sano variant.

U.S. Pat. Nos. 5,797,879 and 6,053,891, to DeCampli, both of which are hereby incorporated by reference, attempted to obviate some of the hemodynamic problems and complications associated with the standard Norwood operation by providing an adjustable constriction on the shunt. The devices described in those patents permit post-operative adjustment of the blood flow through the shunt used in the first stage operations for a variety of congenital heart anomalies.

The devices disclosed in the foregoing patents include a rigid sheath inside which a balloon is mounted eccentrically. The sheath is disposed around the outside of a synthetic vascular graft at the end of the first stage operation. A catheter coupled to the balloon is either brought out through the skin, or connected to a subcutaneous access port. A clinician may adjust blood flow through the shunt by graded inflation or deflation of the eccentric balloon, which externally compresses the shunt. At the end of the period of hemodynamic instability (24-72 hours), or at the second stage operation, the device may be routinely removed.

U.S. Pat. No. 4,256,094 to Kapp et al. describes an arterial pressure control system including an inflatable cuff that encircles an artery. A fluid pump is coupled to the cuff to periodically inflate the cuff responsive to a programmable controller. The controller is programmed to provide a desired pressure in the artery based on a difference between the desired pressure and a signal from a pressure sensor that contacts the artery downstream from the cuff. The patent describes that the controller regulates the output pressure of the pump to inflate or deflate the cuff as needed to maintain the desired pressure.

The device described in the foregoing patent has several drawbacks that render it unsuitable for use in treating HLHS. For example, the pressure sensor described in that patent monitors a pressure level within an artery, not a degree of constriction applied by the cuff to the artery.

Previously-known blood flow redistribution devices also have employed the principle of “counterpulsation,” wherein a vessel is periodically occluded to augment or otherwise regulate blood flow. For nearly three decades, surgeons have used intraaortic balloon counterpulsation to augment coronary blood flow in adults with ischemic heart disease (coronary artery disease).

A typical counterpulsation device (e.g., such as sold by DataScope, Inc.), often called an “intraaortic balloon pump” (IABP), includes a catheter having a long (8-20 cm) balloon distal on its distal region. The catheter is advanced intravascularly from an access site, e.g., in the femoral artery, so that the balloon is positioned in the proximal descending thoracic aorta. The catheter is connected to a control unit that periodically inflates the balloon with carbon dioxide gas at the start of cardiac diastole, and deflates the balloon with the start of systole, as detected using the ECG or arterial blood pressure trace. Such devices have long been shown to augment diastolic coronary blood flow and improve cardiac output in older children and adults with coronary artery disease, and to improve survival in adult patients with cardiogenic shock.

Although intended for temporary use (up to about one week), at least one IABP is designed for long-term use or permanent implantation within the aorta. The Kantrowitz Cardio VAD™, available from L.VAD Technology, Inc., Detroit, Mich., is implanted by opening the aorta and sewing a Dacron cuff into the aortic wall. The balloon then is attached to the inner wall of the Dacron cuff. In a thirty-day trial of the device, there were no strokes or other thromboembolic events.

Previously-known systems also are known in which counterpulsation techniques were implemented by applying external compression to a vessel. For example, a 1976 report describes laboratory use of a flexible pneumatic pumping chamber of polyurethane encased in an ellipsoid Dacron graft, which was wrapped around the descending thoracic aorta. In 2002, a new method to achieve aortic counterpulsation in animals was reported in which a similar device was positioned around the outside of the ascending aorta. Such demonstrations have established that extravascular counterpulsation may be as effective as intravascular counterpulsation in improving hemodynamics in open chest sheep, however, these techniques have found limited acceptance for use in humans.

Still other methods of counterpulsation are known. For example, “enhanced external counterpulsation” (EECP) provides counterpulsation of the peripheral vessels by inflation and deflation of cuffs wrapped around a patient's extremities. Such devices, however, could not be applied to a synthetic graft.

In view of the foregoing, it would be desirable to provide apparatus and methods for use in treating patients with HLHS that avoids the potential for kinking or distortion of a right ventricular shunt or of a pulmonary vessel as encountered in the Sano variant.

It also would be desirable to provide apparatus and methods for use in treating patients with HLHS and other pediatric cardiac abnormalities that mitigate the risk of coronary artery ischemia arising from conventional shunting techniques.

It further would be desirable to provide apparatus and methods that obviate attachment of a shunt directly to the single pumping heart chamber of a patient suffering from HLHS.

It further would be desirable to provide apparatus and methods that may be used in conjunction with a synthetic vascular shunt to provide post-operative adjustment of flow through the shunt while reducing the need for reinterventions.

It further would be desirable to provide apparatus and methods that may be applied to pediatric patients to regulate the flow of blood between the lungs and the remainder of the patient's body, while reducing the risk of cardiac ischemia and shunt or vessel distortion.

### SUMMARY OF THE INVENTION

In view of the foregoing, it is an object of the present invention to provide apparatus and methods for use in treating patients with HLHS that avoids the potential for kinking or distortion of a right ventricular shunt or of a pulmonary vessel as encountered in the Sano variant.

It is also an object of this invention to provide apparatus and methods for use in treating patients with HLHS and other pediatric cardiac abnormalities that mitigate the risk of coronary artery ischemia arising from conventional shunting techniques.

It is further object of the present invention to provide apparatus and methods that obviate attachment of a shunt directly to the single pumping heart chamber of a patient suffering from HLHS.

It is another object of this invention to provide apparatus and methods that may be used in conjunction with a synthetic vascular shunt to provide post-operative adjustment of flow through the shunt while reducing the need for reinterventions.

It still further is an object of the present invention to provide apparatus and methods that may be applied to pediatric patients to regulate the flow of blood between the lungs and the remainder of the patient's body, while reducing the risk of cardiac ischemia and shunt or vessel distortion.

It is also an object of the present invention to provide apparatus and methods that monitor a degree of flow regulation through a shunt responsive to blood pressure fluctuations arising from cardiac activity.

This and other objects of the invention are accomplished by providing a shunt for use in treating HLHS and other cardiac abnormalities wherein the shunt includes an extravascular counterpulsation capability, thereby providing the short-term hemodynamic advantages of the Sano variant, while avoiding the potential complications of that technique. Apparatus constructed in accordance with the principles of the present invention allows a surgeon to perform a standard Norwood operation (with a synthetic shunt coupled between the aortic arch and the pulmonary vessels), while providing a mechanical way to alter the blood flow through the shunt in a desired manner. Specifically, the apparatus occludes, or partially occludes flow through the shunt during diastole, so as to impede diastolic pulmonary runoff and increase diastolic blood pressure. The result is an augmented coronary blood flow reserve in diastole, greater hemodynamic stability in the early post-operative period, and greater survival rate.

By applying extravascular counterpulsation techniques only to the synthetic shunt, the potential for vessel wall trauma encountered with previously-known intravascular and extravascular counterpulsation systems may be avoided. Moreover, the configuration of the apparatus permits ready application in infants and children, as well as adults, thereby reducing pulmonary runoff and augmenting diastolic perfusion of the coronary arteries.

In one embodiment, the apparatus comprises an implantable portion and an external portion. The implantable portion comprises a synthetic vascular graft and an implantable cuff configured to be implanted in apposition to an exterior wall of the graft. The implantable cuff includes an expandable element configured to selectively constrict the flow area of the graft. The external portion comprises a controller, an inflator coupled to the expandable element for periodically inflating and deflating the expandable element responsive to an output of the controller, and a sensor coupled to the controller that provides a signal corresponding to the cardiac activity of the patient. The controller is programmed to actuate the inflator to adjust a degree of constriction applied by the expandable element in synchrony with the cardiac activity of the patient.

Alternatively, the graft and cuff portions of the implantable portion may be integrally formed to reduce the size of the implantable portion, thereby permitting use of the device in even smaller patients.

Methods of using the apparatus of the present invention to regulate blood flow in aortopulmonary shunts also are provided.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a perspective view of a heart following completion of a conventional Norwood operation to palliate HLHS;

FIG. 2 is a schematic view of apparatus of the present invention;

FIGS. 3A and 3B are, respectively, perspective and exploded views of a first illustrative embodiment of an implantable portion of the present invention; and

FIG. 4 is a side sectional view of an implantable portion of an alternative embodiment of the apparatus of the present invention.

### DETAILED DESCRIPTION OF THE INVENTION

The present invention is directed to methods and apparatus for treating HLHS and other cardiac abnormalities, especially those in pediatric patients. Conventional methods for treating HLHS typically involve three stages of operations, beginning with the Norwood operation. FIG. 1 depicts a HLHS heart upon completion of a typical Norwood operation. In this first stage operation, synthetic vascular graft 10 is coupled between innominate artery IA (brachiocephalic trunk) originating from aortic arch AA and pulmonary arteries PA. Also during this operation, the pulmonary arteries are disconnected from the pulmonary trunk PT, and patch 12 placed to close the vessel. The underdeveloped aortic arch AA is expanded with graft 14 and coupled to the pulmonary trunk. In addition, atrial septum AS is removed, so that blood returning from the lungs through pulmonary veins PV exits into the right atrium RA.

As noted above, it has been observed that the shunt may induce “pulmonary runoff” during diastole, in which blood is siphoned from the coronary arteries (not shown) into pulmonary arteries PA, leading to reduced blood pressure in the coronary arteries and ventricular ischemia. While the Sano variant relieves the problem arising from such runoff, by connecting shunt 10 directly between the ventricle and pulmonary arteries PA, that relatively new method has other potential complications, which are not yet fully understood.

The present invention addresses these drawbacks of previously known methods and apparatus by applying extravascular counterpulsation techniques to a conventional aortopulmonary shunt. The apparatus and methods of the present invention thereby are expected to reduce pulmonary runoff and augment diastolic perfusion of the coronary arteries, enhancing the patient's odds of survival.

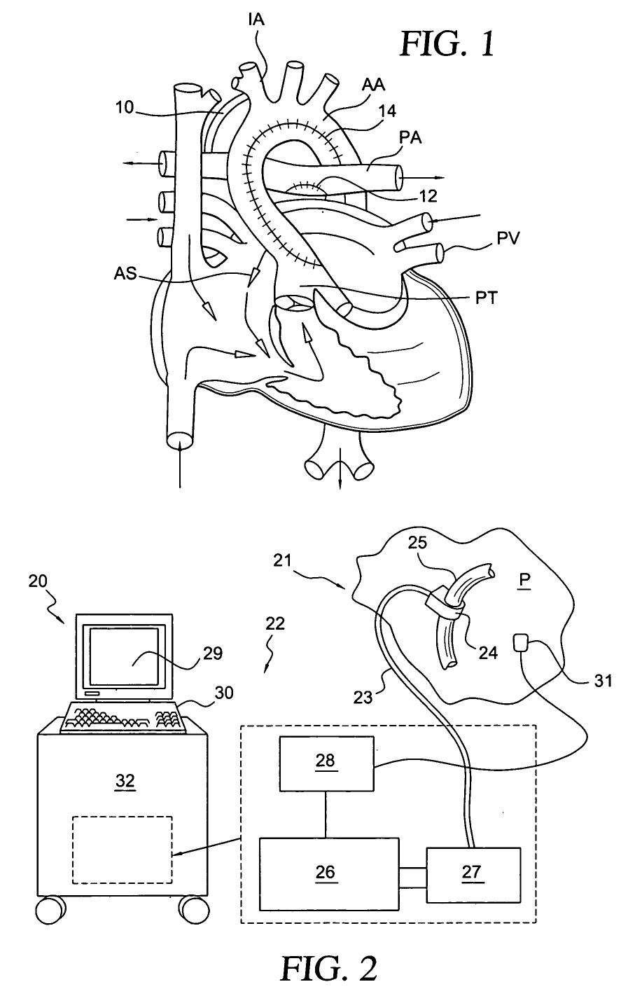

Referring now to FIG. 2, apparatus 20 of the present invention is described. Apparatus 20 comprises implantable portion 21 and external portion 22, coupled by flexible tube 23. Implantable portion 21 comprises cuff 24 disposed to surround a portion of the exterior of shunt 25. Shunt 25 comprises a synthetic polymeric material, e.g., GORTEX® (polytetrafluoroethylene), and for treatment of HLHS is configured as an aortopulmonary shunt, such as depicted in FIG. 1. In accordance with the principles of the present invention, cuff 24 includes an expandable element, e.g., a balloon that may be periodically inflated and deflated to vary the internal flow area of shunt 25.

External portion 22 of apparatus 20 comprises controller 26, inflator 27, cardiac activity detector 28, display 29 and input device 30. Cardiac activity detector 28 is coupled to sensor 31, which is applied to patient P. Illustratively, external portion 22 may be housed in cart 32 or mounted on an IV-pole so that inflator 27 may be placed in proximity to the patient and coupled via flexible tube 23 to implantable portion 21.

The expandable element of cuff 24 is in fluid communication with inflator 27, which periodically inflates and deflates the expandable element using a suitable biocompatible fluid or gas via flexible tube 23. Inflator 27 is actuated responsive to an output signal generated by controller 26, which in turn is synchronized to the patient's cardiac activity. In accordance with the principles of the present invention, cuff 24 is configured to operate in a counterpulsating manner, inflating cuff 24 during diastole and deflating the cuff during systole. In this manner, blood is permitted to flow through shunt 25 during systole, but is constricted to reduce pulmonary runoff during diastole.

Controller 26 is coupled to receive a signal from cardiac activity detector 28 that informs the controller of the phase of the patient's cardiac cycle. Cardiac activity detector 28 may constitute, for example, an EKG detector using one or more sensors 31, or an output of an arterial pressure monitor. Controller 26 is programmed to actuate inflator 27 responsive to the signal from cardiac activity detector 28 to modulate the degree of constriction induced in shunt 25 by cuff 24.

For example, controller 26 may be programmed to actuate inflator 27 at a given point of the cardiac cycle, so that the shunt is constricted to a predetermined degree at the onset of diastole. Following contraction of the ventricle, the ventricular pressure typically falls below aortic pressure about two-thirds of the way through the T-wave of the cardiac cycle. Thus, controller 26 may be programmed to actuate inflator 27 upon detection of the onset of the T-wave, so that shunt 25 is fully constricted at the onset of diastole. Likewise, controller 26 may be programmed to actuate inflator 27 to relieve pressure in cuff 24 upon detection of the P-wave of the cardiac cycle, so that shunt 25 is open to its maximum extent prior to the onset of systole. Alternatively, if the cardiac activity detector comprises an arterial pressure monitor, controller 26 may be programmed to respond directly to threshold pressure levels.

It should of course be understood that the initiation points for actuation of inflator 27, time intervals of actuation and inflator pressure 27 all may be programmable controlled by controller 26 using input device 30. In addition, cardiac activity detected by cardiac activity detector 28 and other parameters of interest, e.g., arterial pressure or a computed degree of constriction of shunt 25, may be displayed on display 29.

The foregoing arrangement enables controller 26 to detect a phase of the cardiac cycle and initiate inflation of the expandable element at the beginning of diastole and deflation at the beginning of systole. Because cuff 24 impedes pulmonary flow during diastole, it may be desirable to employ a slightly larger diameter (0.5 cm larger) graft for shunt 10 to augment pulmonary flow during systole. Apparatus 20 preferably is designed to operate with a cycle time as short as 300 ms.

External portion 22 preferably includes numerous safety measures to reduce the risk of improper operation of cuff 24. For example, controller 26 may be programmed to prevent the expandable element of cuff 24 from remaining in an inflated position beyond a certain time interval. More specifically, controller 26 may include a timer circuit or programming that detects a period of inflation for cuff 24. If the period of inflation exceeds a predetermined time, controller 26 may initiate an automatic deflate mode. Alternatively, if the cuff remains in an inflated state after the automatic deflate mode has been initiated, controller 26 may activate an alarm to alert a clinician to manually actuate inflator 27 to relieve the pressure, or to cut flexible tube 23.

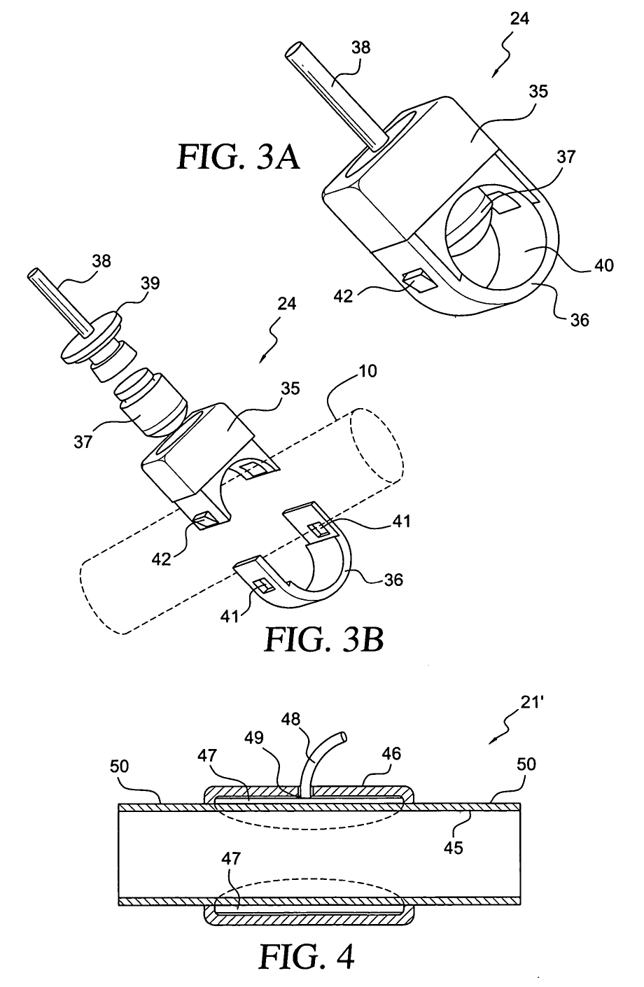

Referring now to FIGS. 3A and 3B, further details of cuff 24 of implantable portion 21 of the embodiment of FIG. 2 are described. Cuff 24 preferably comprises housing 35 having U-shaped element 36, balloon 37, and inflation tube 38. Housing 35 preferably comprises a substantially rigid biocompatible plastic molded or machined to a size suitable for use in an intended application, and may include anchor 39 that fastens balloon 37 within the housing.

For pediatric use, housing 35 may have a length of approximately 10 mm. U-shaped element 37 couples housing 35 to shunt 10 (shown in dotted outline in FIG. 3B), so that balloon 37 contacts the exterior surface of the shunt. Housing 35 and U-shaped element 36 when engaged define aperture 40 through cuff 24 having a diameter slightly larger than that of shunt 10. Aperture 40 preferably should have a diameter in a range of 2.5 to 6.0 mm for use on shunts for treating HLHS.

Cuff 24 of FIG. 3 is configured to be engaged with aortopulmonary shunt 10 just prior to completion of the Norwood operation, by clamping U-shaped element 36 to housing 35 so that the cuff surrounds the exterior of a selected portion of shunt 10. Housing 35 and U-shaped element 36 preferably have apertures 41 that engage detents 42 formed on the exterior surface of housing 35, so the housing 35 and element 36 snap together to encircle shunt 10. It will of course be understood that other suitable retaining elements may be used to lockingly interengage U-shaped element 36 to housing 35. Advantageously, cuff 24 is disposed entirely extravascularly.

Balloon 37 preferably comprises a compliant or semi-compliant biocompatible material, such as nylon or polyurethane, and is inflated through inflation tube 38, which may be coupled to flexible tube 23 via a suitable connector. Balloon 37 should be sufficiently robust to be subjected to the expected number of inflation and deflation cycles for the intended application, and may be inflated with a suitable biocompatible, relatively chemically inert fluid or gas, such as saline, helium or carbon dioxide. For example, balloon 37 may be designed, using known techniques, to precisely inflate to, and rapidly deflate from, a prescribed volume such as approximately 1 cc, with a cycle time of about 0.3 sec and for a period of up to 72 hours.

Balloon 37 is firmly attached within the housing 35 so that upon inflation the balloon expands substantially in a radially direction into aperture 40. Balloon 37 may be measured at a series of inflation volumes during manufacture to empirically derive a formula that relates inflation volume to the degree of constriction of a shunt disposed within aperture 40, which also may vary as a function of the pressure within the shunt. This relationship may be programmed into controller 24 for each cuff 24, so that a predetermined interval of actuation of inflator 27 will provide a predictable degree of constriction of the shunt. In particular, the controller may be programmed to inflate the balloon to such a diameter as to ensure complete occlusion of the shunt.

Inflation tube 38 is attached to balloon 37 and may extend a predetermined distance, for example, 5-10 cm, from housing 35, thereby facilitating access to cuff 24. The inflation tube may exit through the skin of a patient to be coupled to flexible tube 23, or may be attached to a subcutaneous port that may be subsequently accessed using a small caliber needle. Inflation tube 38 may be secured to anchor 39, which may in turn be secured to housing 35 by threads or any other suitable form of retaining element or connector.

Referring now to FIG. 4, an alternative embodiment of an implantable portion constructed in accordance with the present invention is described. Implantable portion 21′ of FIG. 4 is similar in construction to the grafts described in commonly owned U.S. Pat. No. 5,797,879, and may be substituted for shunt 10 and cuff 24 of the embodiment of FIG. 2.

Implantable portion 21′ includes synthetic graft 45 enclosed within substantially rigid sheath 46. Toroidal balloon 47 is disposed within sheath 46 and encircles graft 45. Inflation tube 48, which extends through aperture 49 in sheath 46, is coupled at one end to balloon 47 and may be coupled at the other to flexible tube 23 of the apparatus of FIG. 2. When inflated by inflator 27, balloon 47 urges the encircled portion of graft 45 radially inward, as depicted in dotted lines in FIG. 4, thereby constricting the internal flow area of the shunt.

Implantable portion 21′ of FIG. 4 is intended to streamline the process of assembling cuff 24 to the exterior of shunt 10, as in the embodiment of FIG. 3, by providing an integrated graft and cuff for use as the aortopulmonary shunt. Accordingly, synthetic graft portions 50 disposed on either side of sheath 46 may be cut to size and implanted during the Norwood operation. Advantageously, implantable portion 21′ may obviate difficulties associated with attaching the cuff to the shunt, and may provide a more accurate correlation between inflation volume and degree of shunt constriction.

Methods of using the apparatus of the present invention, such as that depicted in FIG. 2, are now described. Typically, cuff 24 is affixed to the exterior of an aortopulmonary shunt prior to completion of a Norwood operation. Once cuff 24 is implanted, it is coupled to flexible tube 23 and inflator 27. Sensor 31 is applied to the patient, and then external portion 22, including controller 26, inflator 27 and cardiac activity detector 28, are powered up and tested. Using input device 30, the clinician then inputs a profile of expected values, e.g., degree of constriction, inflation interval and pressure, etc., to control operation of apparatus 20.

Responsive to detected cardiac activity, such as electrocardiogram (ECG) signal or arterial pressure trace generated using sensor 31, detector 28 outputs a signal to controller 26. Controller 26 in turn controls operation of inflator 27 to periodically adjust the degree of constriction of shunt 10, and thereby reduce diastolic pulmonary runoff. The monitored cardiac activity and a trace representative of the cuff inflation status may be displayed on display 29. The clinician may then uses that displayed information and input device 30 to fine tune the parameters controlling operation of the controller 26.

Controller 26 also may monitor whether inflator 27 and cuff 24 are functioning properly. If a determination is made that operation of the inflator 27 is acceptable, no action is taken. If, however, a determination is made that inflator 27 is not functioning properly, e.g., the cuff remains inflated for longer than a preset threshold interval, the inflator may be instructed to deflate the cuff. After activating the deflate mode, a determination is made regarding whether the cuff has successfully deflated, the controller may resume normal operation. If a determination is made that the cuff has not deflated, the controller may activate an audible or visible alarm to indicate that manual corrective action is required. Once the source of the inflation defect is manually corrected, the controller may be reset to resume normal operation.

The patient may be weaned from the operation of apparatus 20 after the patient has recovered from the operation, usually in the first 24-72 hours post-operatively. Weaning may be performed by, for example, triggering the inflation/deflation cycle with every other cardiac cycle, then every third, then every fourth, etc. Eventually, controller 26 and inflator 27 may be powered down.

Cuff 24 may be removed in one of two ways. It is common for cardiac surgeons to leave the sternotomy open during the initial 24-72 hours post-operatively. In this case, when the patient is judged to be hemodynamically stable and not edematous, a second, brief operation is typically performed during which the surgeon closes the chest. Cuff 24 may be removed during this second, brief operation. Alternatively, if the chest already has been closed or it is anticipated that the patient may need adjustment of baseline pulmonary blood flow, the cuff may be connected to a subcutaneous port during the first stage operation and left in place until the second stage operation (typically at 4-6 months of life). The cuff 24 may then be removed during the second stage operation.

Use and operation of implantable portion 21′ of the embodiment of FIG. 4 is similar to that described above for cuff 24. After the weaning period, inflation tube 48 may be sealed with balloon 47 in the deflated state and left in a subcutaneous position until the shunt is removed during the second stage operation.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. Apparatus for regulating blood flow in a patient suffering hypoplastic left heart syndrome or other cardiac anomalies requiring surgical construction of a systemic-to-pulmonary shunt following completion of a first stage palliative procedure, the apparatus comprising:
a synthetic graft configured to be coupled between a patient's systemic and pulmonary vasculature;
a cuff that encircles the synthetic graft extravascularly, the cuff including an expandable element configured to selectively constrict the internal flow area of the synthetic graft;
an inflator coupled to the expandable element; and
a cardiac activity detector that outputs a signal corresponding to cardiac activity of the patient; and
a controller coupled to the inflator and the cardiac activity detector, the controller programmed to actuate the inflator to adjust a degree of constriction imposed on the graft by the expandable element responsive to the signal output by the cardiac activity detector.

2. The apparatus of claim 1 wherein the controller is programmed to cause constriction of the graft during diastole, thereby reducing diastolic pulmonary runoff.

3. The apparatus of claim 1 wherein the controller is programmed to remove constriction of the graft just prior to onset of systole.

4. The apparatus of claim 1 wherein the cuff comprises a housing and a U-shaped element configured to interengage to encircle the graft.

5. The apparatus of claim 4 wherein the housing comprises detents that engage apertures disposed in the U-shaped element.

6. The apparatus of claim 1 wherein the synthetic graft and cuff are integrated to form a unitary implantable portion.

7. The apparatus of claim 6 wherein the expandable element comprises a toroidal balloon.

8. The apparatus of claim 1 further comprising a display and an input device.

9. The apparatus of claim 1 wherein the inflator is capable of operating with a cycle time of 300 milliseconds.

10. The apparatus of claim 1 wherein the expandable element is a balloon and the inflator inflates the balloon with a biologically and chemically inert gas or fluid.

11. A method of controlling blood flow in a patient suffering hypoplastic left heart syndrome or other congenital cardiac anomalies in which a surgically constructed systemic-to-pulmonary shunt is required following completion of a first stage palliative procedure, the method comprising:
implanting an aortopulmonary graft;
providing an implantable cuff having an expandable element and an inflation tube disposed in fluid communication with an interior of the expandable element;
disposing the cuff in an encircling relation about an exterior of the aortopulmonary graft;
coupling the inflation tube to an inflator configured to selectively actuate the expandable element to constrict an internal flow area of the graft;
detecting cardiac activity of the patient; and
activating the inflator based on the detected cardiac activity.

12. The method of claim 11 further comprising activating the inflator to cause constriction of the graft during diastole and reduce diastolic pulmonary runoff.

13. The method of claim 12 further comprising activating the inflator to remove constriction of the graft just prior to onset of systole.

14. The method of claim 1 wherein disposing the cuff in an encircling relation comprises interengaging a U-shaped element to a housing to encircle the graft.

15. The method of claim 14 wherein interengaging the U-shaped element to the housing further comprises engaging apertures in the U-shaped element with detents disposed on an exterior surface of the housing.

16. The method of claim 11 wherein the synthetic graft and cuff are provided as a unitary implantable portion.

17. The method of claim 11 further comprising actuating the inflator with a cycle time of 300 milliseconds or less.

18. The method of claim 11 further comprising determining a status of the expandable element and activating an alarm if the expandable element remains inflated beyond a threshold interval.

19. The method of claim 18 wherein activating an alarm comprises generating at least one of a visible alarm and an audible alarm.

20. The method of claim 11 further comprising displaying detected cardiac activity and a parameter corresponding to a degree of constriction of the graft.
